(19) United States
(12) Patent Application Publication
(10) Pub. No.: US 2014/0276038 A1
(43) Pub. Date: Sep. 18, 2014

(54) SYSTEMS AND METHODS FOR PREDICTING IMPACT OF A CATHETER ON CURVATURE OF A VESSEL

(71) Applicant: VOLCANO CORPORATION, San Diego, CA (US)

(72) Inventors: Joseph Burnett, Carlsbad, CA (US); K. Matthews, San Diego, CA (US); Neil Hattangadi, San Diego, CA (US); Scott Huennekens, San Diego, CA (US); Bret Millett, Folsom, CA (US)

(73) Assignee: VOLCANO CORPORATION, San Diego, CA (US)

(21) Appl. No.: 14/207,778

(22) Filed: Mar. 13, 2014

Related U.S. Application Data
(60) Provisional application No. 61/778,795, filed on Mar. 13, 2013.

(51) Int. Cl. A61B 6/00; G06N 5/04

(52) U.S. Cl. CPC A61B 6/481; G06N 5/04
USPC 600/431; 706/46

(57) ABSTRACT

The invention generally relates to systems and methods for predicting impact of a catheter on curvature of a vessel. In certain aspects, the invention provides a system for predicting impact of a catheter on curvature of a vessel. The system includes a central processing unit (CPU) and storage coupled to the CPU for storing instructions. The stored instructions, when executed by the CPU, cause the CPU to accept as input data representative of stiffness of a catheter. The CPU is additionally caused to compare the data to a material parameters data set. The material parameters data set includes vessel displacement values for catheters of different stiffness. The CPU is additionally caused to predict impact of the catheter on a curvature of a vessel based on the comparison of the data to the material parameters data set, and to provide the prediction as an output.

### RELATED APPLICATION

The present application claims the benefit of and priority to U.S. provisional patent application Ser. No. 61/778,795, filed Mar. 13, 2013, the content of which is incorporated by reference herein in its entirety.

### FIELD OF THE INVENTION

The invention generally relates to systems and methods for predicting impact of a catheter on curvature of a vessel.

### BACKGROUND

Intravascular imaging and endovascular surgery have increased the life expectancy and quality of life for patients suffering from cardiovascular disease. Imaging techniques such as intravascular ultrasound (IVUS), intravascular Doppler, and intravascular optical coherence tomography (OCT) allow radiologists, neurologists, neurosurgeons, cardiologists, vascular surgeons, etc., to directly visualize a patient's vasculature to observe occlusions, thrombi, embolisms, aneurisms, etc. Coupling the imaging techniques with advanced surgical procedures, it is possible to counteract cardiovascular disease by removing thrombi or placing stents in weakened vessels. Using such procedures, a patient at high risk for cardiac arrest can have the risk lessened, and experience a better quality of life after treatment. Furthermore, because intravascular imaging and endovascular surgery are less invasive than techniques such as coronary bypass, the risk of surgical complication is greatly reduced and hospital stays and recovery times are shortened.

While the procedures are non-invasive, the substantial distance between the entry into the body and the targeted tissue makes the procedures complex. Angiography images are typically used to assist in getting the intravascular imaging system to the treatment site. Images of the vessel and treatment area are acquired prior to a procedure and used to guide the procedure. A problem with this approach is that the angiography images static images that are taken prior to the intravascular imaging catheter reaching the treatment site. Accordingly, those images do not inform an operator how the catheter itself will affect the vessel. For example, static angiography images do not account for how the curvature of a vessel will change in response to the stiffness of the catheter being inserted into the vessel.

To address that problem, live angiography can be used, in which real-time imaging is performed in connection with the intravascular imaging procedure. Unfortunately, live angiography presents risks to both the patient and the provider. Live angiography uses a fairly continuously flow of radiopaque contrast agents and continuous x-ray imaging, e.g., fluoroscopy, to image the vasculature. Because the images are taken in real time, substantially greater amounts of x-ray radiation are required as compared to a radiograph (x-ray picture). In addition to the x-ray exposure, patients may suffer side effects from the radiopaque contrast agents, including pain, adverse drug interactions, and renal failure. For technicians and physicians, there are also risks of x-ray exposure as well as orthopedic injuries (e.g., lower back strain) due to the extra weight of the lead-lined aprons and other protective equipment.

### SUMMARY

The invention provides systems and methods that can predict the impact that a catheter will have on a vessel, such as the curvature of the vessel. Accordingly, systems and methods of the invention allow interventional and intravascular imaging procedures to be conducted using static images, with little or no need to update those images using live angiography. Aspects of the invention are accomplished using a material parameters data set. The material parameters data set includes vessel displacement values for catheters of different stiffness. Data representative of stiffness of a catheter is compared to the material parameters data set. The impact of the catheter on a curvature of a vessel is predicted based on the comparison of the data to the material parameters data set. Once the data is matched to a value in the material parameters data set, the prediction is output. That output may be displayable on a display device.

In certain aspects, the invention provides a system for predicting the impact that a catheter will have on a vessel. The system includes a central processing unit (CPU) and storage coupled to the CPU for storing instructions. The stored instructions, when executed by the CPU, cause the CPU to accept as input data representative of stiffness of a catheter. The CPU is additionally caused to compare the data to a material parameters data set. The material parameters data set includes vessel displacement values for catheters of different stiffness. The CPU is additionally caused to predict impact of the catheter on a curvature of a vessel based on the comparison of the data to the material parameters data set, and provide the prediction as an output. The material parameters data set may be stored at a location remote from the system and received by the system prior to the comparison.

In other aspects, the invention provides methods for predicting impact of a catheter on curvature of a vessel. The methods involve accepting as input data representative of stiffness of a catheter. Methods of the invention additionally involve comparing the data to a material parameters data set that includes vessel displacement values for catheters of different stiffness. Methods of the invention additionally involve predicting impact of the catheter on a curvature of a vessel based on the comparison of the data to the material parameters data set, and providing the prediction as an output.

Each value may be generated by obtaining computed tomography angiography images, co-registering those images to live angiograms, measuring the vessel displacement with and without catheter insertions of different stiffness, and linking the displacements to non-invasive anatomic anchor points identified in the images. Methods co-registering image data are described, for example in Huennekens et al. (U.S. patent application number 2013/0030295), the content of which is incorporated by reference herein its entirety. In certain embodiments, the non-invasive anatomic anchor points are automatically identified. Systems and methods of the invention may be used with any vessels. In certain embodiments, the vessel is of the cardiovascular system.

The invention also provides another configuration of a system for predict the impact that a catheter will have on a vessel. The system includes a central processing unit (CPU) and storage coupled to the CPU for storing instructions. The stored instructions, when executed by the CPU, cause the CPU receive a request from a computer for a stored material parameters data set that includes vessel displacement values for catheters of different stiffness. The CPU is additionally caused to provide the computer with the material parameters data set to allow the computer to compare data representative of stiffness of a catheter to the material parameters data set, and provide the prediction as an output from the computer. The material parameters data set may be stored at a location remote from the computer and received by the computer prior to the comparison.

Another aspect of the invention provides methods for predicting impact of a catheter on curvature of a vessel. the methods involve receiving a request from a computer for a stored material parameters data set that comprises vessel displacement values for catheters of different stiffness, and providing the computer with the material parameters data set to allow the computer to compare data representative of stiffness of a catheter to the material parameters data set, and provide the prediction as an output from the computer.

### BRIEF DESCRIPTION OF THE DRAWINGS

FIG. 1 illustrates a system for performing methods of the invention.

FIG. 2 is a process chart depicting the procedural steps for predicting impact of a catheter on curvature of a vessel, according to certain embodiments.

### DETAILED DESCRIPTION

The invention generally relates to systems and methods for predicting impact of a catheter on curvature of a vessel. In certain aspects, the invention provides a system for predicting impact of a catheter on curvature of a vessel. The system includes a central processing unit (CPU) and storage coupled to the CPU for storing instructions. The stored instructions, when executed by the CPU, cause the CPU to accept as input data representative of stiffness of a catheter. The CPU is additionally caused to compare the data to a material parameters data set. The material parameters data set includes vessel displacement values for catheters of different stiffness. The CPU is additionally caused to predict impact of the catheter on a curvature of a vessel based on the comparison of the data to the material parameters data set, and to provide the prediction as an output.

The material parameters data set is generated by associating catheter stiffness with vessel displacement caused by the catheter. Material parameters of the catheter, such as, the material that makes-up the catheter, the thickness of the catheter, and the size of the catheter (i.e., inner and outer diameter) are obtained. The stiffness of the catheter is then measured. Methods for measuring catheter stiffness are known in the art. Exemplary methods are described for example in Stenqvist et al. (Acta Anaesthesiol Scand., 2:153-157, 1983), the content of which is incorporated by reference herein in its entirety. The stiffness value is associated with the recorded parameters for that catheter.

Once the parameters and stiffness of a catheter are obtained and associated, the impact of that catheter on a vessel is measured. The measurement is performed by obtaining computed tomography angiography images and co-registering those images to live angiograms. Methods co-registering image data are described, for example in Huennekens et al. (U.S. patent application number 2013/0030295), the content of which is incorporated by reference herein in its entirety.

Then, the vessel displacement is measured with and without catheter insertions of different stiffness. Those displacements are linked to non-invasive anatomic anchor points identified in the images. Linking across different images is described for example in Vince et al. (U.S. Pat. No. 6,200,268), the content of which is incorporated by reference herein in its entirety. From the computed tomography angiography images, an operator selects a region of interest in the computed tomography angiography image. The x,y coordinates defining the region of interest are determined from the computed tomography angiography images. The coordinates are then mapped to the same location in the corresponding live angiogram and an live angiogram region of interest that corresponds to the computed tomography angiography region of interest is determined. Displacement of the vessel within the live angiograph caused by catheter insertion is measured. That displacement is translated to the computed tomography angiography and the displacement is associated with an anatomic anchor point in the computed tomography angiography image. The above-described process is repeated for each region of each for each catheter and repeated for each component as many times as desired in order to obtain a more accurate range of signal properties characteristic of the component.

With the database built, given a set of parameters of a catheter, those parameters can automatically and accurately be used to predict vessel displacement caused by the catheter. Accordingly, one can guide interventional and intravascular imaging procedures using static images, with little or no need to update those images using live angiography.

Aspects of the invention described herein can be performed using any type of computing device, such as a computer, that includes a processor, e.g., a central processing unit, or any combination of computing devices where each device performs at least part of the process or method. In some embodiments, systems and methods described herein may be performed with a handheld device, e.g., a smart tablet, or a smart phone, or a specialty device produced for the system.

Methods of the invention can be performed using software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections).

Processors suitable for the execution of computer program include, by way of example, both general and special purpose microprocessors, and any one or more processor of any kind of digital computer. Generally, a processor will receive instructions and data from a read-only memory or a random access memory or both. The essential elements of computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, (e.g., EPROM, EEPROM, solid state drive (SSD), and flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having an I/O device, e.g., a CRT, LCD, LED, or projection device for displaying information to the user and an input or output device such as a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The subject matter described herein can be implemented in a computing system that includes a back-end component (e.g., a data server), a middleware component (e.g., an application server), or a front-end component (e.g., a client computer having a graphical user interface or a web browser through which a user can interact with an implementation of the subject matter described herein), or any combination of such back-end, middleware, and front-end components. The components of the system can be interconnected through network by any form or medium of digital data communication, e.g., a communication network. For example, the reference set of data may be stored at a remote location and the computer communicates across a network to access the reference set to compare data derived from the female subject to the reference set. In other embodiments, however, the reference set is stored locally within the computer and the computer accesses the reference set within the CPU to compare subject data to the reference set. Examples of communication networks include cell network (e.g., 3G or 4G), a local area network (LAN), and a wide area network (WAN), e.g., the Internet.

The subject matter described herein can be implemented as one or more computer program products, such as one or more computer programs tangibly embodied in an information carrier (e.g., in a non-transitory computer-readable medium) for execution by, or to control the operation of, data processing apparatus (e.g., a programmable processor, a computer, or multiple computers). A computer program (also known as a program, software, software application, app, macro, or code) can be written in any form of programming language, including compiled or interpreted languages (e.g., C, C++, Perl), and it can be deployed in any form, including as a stand-alone program or as a module, component, subroutine, or other unit suitable for use in a computing environment. Systems and methods of the invention can include instructions written in any suitable programming language known in the art, including, without limitation, C, C++, Perl, Java, ActiveX, HTML5, Visual Basic, or JavaScript.

A computer program does not necessarily correspond to a file. A program can be stored in a file or a portion of file that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

A file can be a digital file, for example, stored on a hard drive, SSD, CD, or other tangible, non-transitory medium. A file can be sent from one device to another over a network (e.g., as packets being sent from a server to a client, for example, through a Network Interface Card, modem, wireless card, or similar).

Writing a file according to the invention involves transforming a tangible, non-transitory computer-readable medium, for example, by adding, removing, or rearranging particles (e.g., with a net charge or dipole moment into patterns of magnetization by read/write heads), the patterns then representing new collocations of information about objective physical phenomena desired by, and useful to, the user. In some embodiments, writing involves a physical transformation of material in tangible, non-transitory computer readable media (e.g., with certain optical properties so that optical read/write devices can then read the new and useful collocation of information, e.g., burning a CD-ROM). In some embodiments, writing a file includes transforming a physical flash memory apparatus such as NAND flash memory device and storing information by transforming physical elements in an array of memory cells made from floating-gate transistors. Methods of writing a file are well-known in the art and, for example, can be invoked manually or automatically by a program or by a save command from software or a write command from a programming language.

Suitable computing devices typically include mass memory, at least one graphical user interface, at least one display device, and typically include communication between devices. The mass memory illustrates a type of computer-readable media, namely computer storage media. Computer storage media may include volatile, nonvolatile, removable, and non-removable media implemented in any method or technology for storage of information, such as computer readable instructions, data structures, program modules, or other data. Examples of computer storage media include RAM, ROM, EEPROM, flash memory, or other memory technology, CD-ROM, digital versatile disks (DVD) or other optical storage, magnetic cassettes, magnetic tape, magnetic disk storage or other magnetic storage devices, Radiofrequency Identification tags or chips, or any other medium which can be used to store the desired information and which can be accessed by a computing device.

As one skilled in the art would recognize as necessary or best-suited for performance of the methods of the invention, a computer system or machines of the invention include one or more processors (e.g., a central processing unit (CPU) a graphics processing unit (GPU) or both), a main memory and a static memory, which communicate with each other via a bus.

In an exemplary embodiment shown in FIG. 1, system 200 can include a computer 249 (e.g., laptop, desktop, or tablet). The computer 249 may be configured to communicate across a network 209. Computer 249 includes one or more processor 259 and memory 263 as well as an input/output mechanism 254. Where methods of the invention employ a client/server architecture, an steps of methods of the invention may be performed using server 213, which includes one or more of processor 221 and memory 229, capable of obtaining data, instructions, etc., or providing results via interface module 225 or providing results as a file 217. Server 213 may be engaged over network 209 through computer 249 or terminal 267, or server 213 may be directly connected to terminal 267, including one or more processor 275 and memory 279, as well as input/output mechanism 271.

System 200 or machines according to the invention may further include, for any of I/O 249, 237, or 271 a video display unit (e.g., a liquid crystal display (LCD) or a cathode ray tube (CRT)). Computer systems or machines according to the invention can also include an alphanumeric input device (e.g., a keyboard), a cursor control device (e.g., a mouse), a disk drive unit, a signal generation device (e.g., a speaker), a touchscreen, an accelerometer, a microphone, a cellular radio frequency antenna, and a network interface device, which can be, for example, a network interface card (NIC), Wi-Fi card, or cellular modem.

Memory 263, 279, or 229 according to the invention can include a machine-readable medium on which is stored one or more sets of instructions (e.g., software) embodying any one or more of the methodologies or functions described herein. The software may also reside, completely or at least partially, within the main memory and/or within the processor during execution thereof by the computer system, the main memory and the processor also constituting machine-readable media. The software may further be transmitted or received over a network via the network interface device.

Exemplary step-by-step methods are described schematically in FIG. 2. It will be understood that of the methods described in FIG. 1, as well as any portion of the systems and methods disclosed herein, can be implemented by computer, including the devices described above. Image data is collected from the female subject regarding the inside of a vessel 301. This data is then inputted into the central processing unit (CPU) of a computer 302. The CPU is coupled to a storage or memory for storing instructions for implementing methods of the present invention. The instructions, when executed by the CPU, cause the CPU to predict impact of the catheter on a curvature of a vessel based. The CPU provides this determination by comparing input data to the material parameters dataset 303. The reference set of data may be stored locally within the computer, such as within the computer memory. Alternatively, the reference set may be stored in a location that is remote from the computer, such as a server. In this instance, the computer communicates across a network to access the reference set of data. The CPU then provides the prediction as an output.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A system for predicting impact of a catheter on curvature of a vessel, the system comprising:
a central processing unit (CPU); and
storage coupled to said CPU for storing instructions that when executed by the CPU cause the CPU to:
accept as input data representative of stiffness of a catheter;
compare the data to a material parameters data set that comprises vessel displacement values for catheters of different stiffness;
predict impact of the catheter on a curvature of a vessel based on the comparison of the data to the material parameters data set; and
provide the prediction as an output.

2. The system according to claim 1, wherein each value generated by:
obtaining computed tomography angiography images;
co-registering those images to live angiograms;
measuring the vessel displacement with and without catheter insertions of different stiffness; and
linking the displacements to non-invasive anatomic anchor points identified in the images.

3. The system according to claim 2, wherein the non-invasive anatomic anchor points are automatically identified.

4. The system according to claim 1, wherein the vessel is of the cardiovascular system.

5. The system according to claim 1, wherein the material parameters data set is stored at a location remote from the system and received by the system prior to the comparison.

6. The system according to claim 1, wherein the output is displayable on a display device.

7. A method for predicting impact of a catheter on curvature of a vessel, the method comprising:
accepting as input data representative of stiffness of a catheter;
comparing the data to a material parameters data set that comprises vessel displacement values for catheters of different stiffness;
predicting impact of the catheter on a curvature of a vessel based on the comparison of the data to the material parameters data set; and
providing the prediction as an output.

8. The method according to claim 7, wherein each value generated by:
obtaining computed tomography angiography images;
co-registering those images to live angiograms;
measuring the vessel displacement with and without catheter insertions of different stiffness; and
linking the displacements to non-invasive anatomic anchor points identified in the images.

9. The method according to claim 8, wherein the non-invasive anatomic anchor points are automatically identified.

10. The method according to claim 7, wherein the vessel is of the cardiovascular system.

11. The method according to claim 7, wherein the output is displayable on a display device.

12. A system for predicting impact of a catheter on curvature of a vessel, the system comprising:
a central processing unit (CPU); and
storage coupled to said CPU for storing instructions that when executed by the CPU cause the CPU to:
receive a request from a computer for a stored material parameters data set that comprises vessel displacement values for catheters of different stiffness; and
provide the computer with the material parameters data set to allow the computer to compare data representative of stiffness of a catheter to the material parameters data set, and provide the prediction as an output from the computer.

13. The system according to claim 12, wherein the material parameters data set is stored at a location remote from the computer and received by the computer prior to the comparison.

14. A method for predicting impact of a catheter on curvature of a vessel, the method comprising:
receiving a request from a computer for a stored material parameters data set that comprises vessel displacement values for catheters of different stiffness; and
providing the computer with the material parameters data set to allow the computer to compare data representative of stiffness of a catheter to the material parameters data set, and provide the prediction as an output from the computer.

15. The method according to claim 14, wherein the material parameters data set is stored at a location remote from the computer and received by the computer prior to the comparison.
